(19) United States
(12) Patent Application Publication
(10) Pub. No.: US 2023/0277364 A1
(43) Pub. Date: Sep. 7, 2023

(54) ABSORBENT BED PADS WITH ANTI-SLIP BACKING

(71) Applicant: Medline Industries, LP, Northfield, IL (US)

(72) Inventors: James Harvey, Spring City, TN (US); Vincent Hahn, Chicago, IL (US); Caleb Townsend, Davis, IL (US)

(21) Appl. No.: 18/144,882

(22) Filed: May 9, 2023

Related U.S. Application Data
(63) Continuation of application No. 16/295,175, filed on Mar. 7, 2019.

(51) Int. Cl. A61F 5/48; A61G 7/05; B32B 9/02; B32B 5/02

(52) U.S. Cl. CPC A61F 5/485; A61G 7/05; B32B 9/02; B32B 5/022; B32B 2307/726; B32B 2262/0253

(57) ABSTRACT

Disclosed is a fluid absorbent bed pad suitable for absorbing bodily fluids of a patient. The pad includes a backing layer comprising a first polymeric fiber and a second polymeric fiber, the second polymeric fiber being relatively tacky relative to the first polymeric fiber and being present in said backing layer in an amount relatively smaller than the amount of said first fiber and comprising a bicomponent fiber. The presence of the second polymeric fiber will impart a tacky nature to the backing to resist slipping and bunching of the bed pad.

### TECHNICAL FIELD

The disclosure relates generally to disposable absorbent bed pads and methods of making same.

### BACKGROUND

Various types of absorbent bed pads, such as underpads and drypads, are used to collect urine and other bodily fluids of a patient person under medical care by placement of the underpads between bedding and the patient. Such devices are used in many healthcare settings such as critical and non-critical nursing units, labor and delivery rooms, and operating rooms.

Disposable underpads (also known as “chucks”) typically include a layer of a fluid-resistant backing, an absorbent layer, and a fluid-permeable top sheet. Some underpads further include superabsorbent polymers that assist with the absorption of fluids. Examples of such underpads are described in U.S. Pat. No. 8,791,321. Drypads are typically higher-cost items with multiple absorbent layers. These products are intended to provide the patient with better skin protection from harmful fluids.

Conventional underpads typically have a polymeric back sheet or bottom layer, sometimes formed from spunbond polypropylene. The backsheet on conventional underpads can be somewhat slick, and, as a result, such underpads tend to move or migrate under a patient. Thus, it is not uncommon for such underpads to bunch up under the patient and create one or more pressure points that may be harmful to or painful for the patient. This movement and bunching of conventional underpads may also result in displacement of their absorbent core when positioned under the patient, which can result the product not functioning properly to collect urine or other bodily fluids between the patient and the bedding. Conventional drypads, some of which also have a spunbond backing layer, likewise can be somewhat slippery and prone to bunching or displacement. Generally, it is desired to provide a bed pad that mitigates the foregoing.

It has now been found that a bed pad may be prepared using a spunbond backing layer wherein a portion of the spunbond backing layer includes a bicomponent fiber, the bicomponent fiber having a tacky component that increases the coefficient of friction between the fiber and a typical cotton bedsheet. This configuration provides a bed pad with an anti-slip backing that resists undesired movement and bunching. The bicomponent fiber is preferably of a sheath/core construction in which the core component of the fiber is composed of polypropylene and in which the sheath component includes a polyethylene/polypropylene copolymer. The spunbond material may be formed in a multi-bank spunbonding machine in which the bicomponent fiber is emitted from one of the terminal banks of the spunbonding machine.

### BRIEF DESCRIPTION OF THE DRAWINGS

Disclosed herein are embodiments of devices and methods pertaining to bed pads. This description includes drawings, wherein:

FIG. 1 is a perspective view illustrating an exemplary underpad placed on a hospital bed and positioned for absorption of urine.

FIG. 2 illustrates a perspective view of an exemplary absorbent drypad;

FIG. 3 is representational cross-sectional view of the absorbent drypad of shown in FIG. 2.

FIG. 4 is a perspective view of a roll of spunbond material useful as the backing layer for the absorbent drypad shown in FIG. 2.

FIG. 5 is a representation of a spun-bonding process useful in the preparation of a backing layer for a bed pad.

FIG. 6 is a representation of plural types of fibers useful in the preparation of a backing layer for a bed pad.

### DETAILED DESCRIPTION

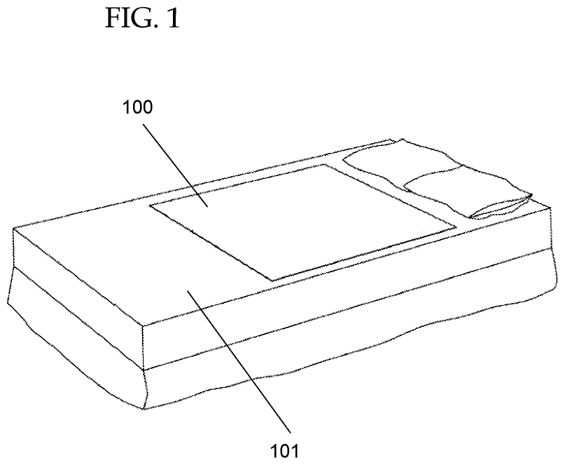

Generally speaking, this disclosure relates to various embodiments of absorbent bed pads, including underpads and methods of making thereof. For example, FIG. 1 illustrates an exemplary absorbent underpad 100 disposed on a bed 101. While the absorbent underpad 100 has been illustrated in FIG. 1 as being generally square in shape, it will be appreciated that the bed pad may be an underpad or bed pad or similar bed pad and substantially of any polygonal shape or any ring-like shape, for example, rectangular, circular, triangular, circular, oval, or of any irregular geometric shape. Generally, the absorbent pad 100 may be of any size suitable for a given use. For example, when placed under a patient between the patient and bedding, the absorbent pad 100 may have a length of about 39-228 cm and a width of about 25-102 cm. Desirable sizes include about 76 cm×91 cm and about 100 cm×230 cm. The bed pad preferably has sufficient cohesive strength to be used to lift at least 90 kg, preferably at least 115 kg, and more preferably at least 135 kg, and even more preferably at least 150 kg.

Generally, the absorbent bed pad 100 may be used for placement under a patient and onto a bedding in any medical care setting to absorb bodily liquids and/or other fluids that may be excreted by the patient. It will be appreciated, however, that the absorbent pad 100 may be used in contexts other than bedding and indeed in non-medical care setting as well, notwithstanding the identification of the pad as a “bed pad.” For example, the pad may serve as an indoor soiling pads for dogs and other household animals, or for use as an absorbent floor mat. The pad may find use in the operating room as a fluid absorbent operating room pad. Associated methods also are contemplated. For example, for use with pets, a suitable method of use generally comprises placing the pad on a surface and allowing a pet to excrete waste onto the pad, then discarding the pad. In the operating room context, the method may comprise placing the pad onto the floor of the operating room or onto a gurney, using the pad to absorb fluids emitted from a patient during a surgical procedure, and then discarding the pad. The pad may also find use in connection with wearable products such as diapers and sanitary napkins.

The bed pad generally comprises an absorbent portion or structure and a backing, the backing intended to provide slip resistance between the bed pad and the bedding onto which the bed pad is placed. The absorbent portion or structure is configured to have the properties of facilitating the drawing-in of liquid, passage of liquid, absorption of liquid, retention of liquid, and the like. The absorbent materials may be natural materials, synthetic materials, and any combination thereof. Generally, the absorbent materials may include, but are not limited to, fibers, foam, fluff, pulp, beads, woven fabrics, nonwoven fabrics, or any combinations thereof.

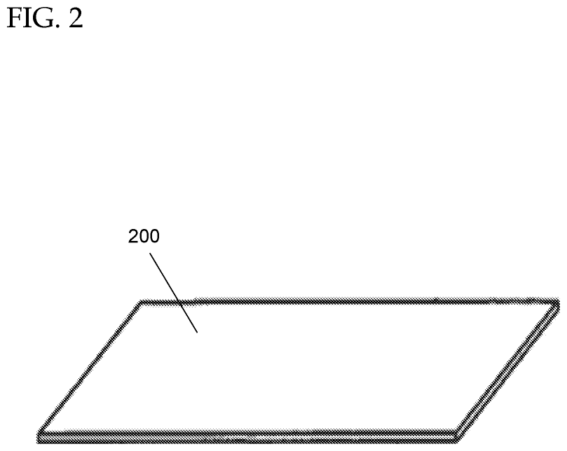

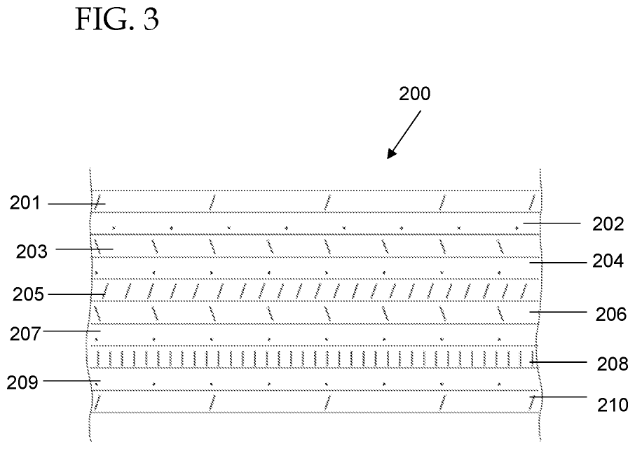

FIG. 2 shows an exemplary absorbent pad 200 that is composes of a laminar structure. With reference to FIGS. 2 and 3, the dry pad 200 includes a spunbond hydrophilic top sheet 201, sheet, typically comprised of polypropylene, a cellulosic tissue layer 203, a super absorbent polymer material 204, a cellulosic pulp layer 205, a second tissue layer 206, a breathable polyethylene film 208, and a spunbond backing material 210, as described in more detail hereinbelow. Adhesive layers 202, 207, and 209 are provided respectively between top sheet 201 and first tissue layer 203, between tissue layer 206 and film 208, and between film 208 and spunbond backing layer 210.

The material of the first layer 201 may comprise any suitable liquid-permeable cover and is preferably a nonwoven material. Suitable nonwoven materials include staple nonwoven materials, melt-blown nonwoven materials, spunmelt nonwoven materials, spunbond nonwoven materials, spunbond meltblown spunbond (SMS) materials, spun lace materials, needle-felted materials, thermal-bonded nonwoven materials, trough-air-bonded nonwoven material, spunlaid nonwoven material, or the like, and any combinations thereof. The material of the first layer 201 may one or more polymeric materials, including, but not limited to, polypropylene, polyethylene, polyethylene terephthalate, polyamide, viscose rayon, nylon, or the like, or any combinations thereof, or may be a biodegradable material such as a starch-based biodegradable material.

Beneath the first layer is an absorbent structure that comprises a cellulosic material, optionally in conjunction with a superabsorbent polymer. These materials may form together a more or less homogeneous layer, or may comprise separate layers. In the illustrated embodiment, the second layer 203 is intended to be a liquid-absorbent layer, and it is preferably a cellulosic tissue layer having a basis weight of 10-20 gsm and a thickness of about 60 to about 100μ. It may comprise a thermally bonded, absorbent air-laid core made from cellulose fibers suitable sources of cellulose include but are not limited to wood fibers, plant fibers, field crop fibers, fluff pulp fibers, cotton, but more generally this layer may be composed of any other material, man-made or natural, that is suitable for absorbing fluid.

Beneath this layer 203 is a layer 204 of superabsorbent polymer, applied to have a basis of 10-20 gsm. Exemplary super absorbent polymers suitable for inclusion into this layer include, but are not limited to, polymers and copolymers of acrylic acid and salts thereof (including alkali metal salts such as sodium salts, or alkaline earth salts thereof), polymers and copolymers of methacrylic acid and salts thereof (including alkali metal salts such as sodium salts, or alkaline earth salts thereof), polyacrylamide polymers and copolymers, ethylene maleic anhydride copolymers, cross-linked carboxy-methyl-celluloses, polyacrylate/polyacrylamide copolymers, polyvinyl alcohol copolymers, cross-linked polyethylene oxides, starch grafted copolymers of polyacrylonitrile, etc. For example, the superabsorbent polymer may comprise sodium polyacrylate.

Beneath the layer 204 of superabsorbent material is disposed a layer 205 of absorbent material. In the illustrated embodiment, the absorbent material is a cellulosic pulp material having a basis of 50-60 gsm and a thickness of about 1.25 to about 1.90 mm. This layer is intended to provide a wicking mechanism. Beneath this layer 204 is another tissue layer 206 that is configured substantially similarly to tissue layer 202.

Beneath this tissue layer 206 is a film layer 208 that is air-permeable and breathable and that is provided to permit air circulation beneath the superseding moisture-absorbent layers and the patient bedding. This layer 208 may comprise any suitable air-permeable material, such as a hydrophobic polymer than includes one or more of polypropylene, polyethylene, polyethylene terephthalate, polyamide, viscose rayon, nylon, or any combinations thereof. This layer may have any suitable a moisture transfer rate, such as a moisture transfer rate ranging from about 5 to about 200 sec/ml, as measured by the moisture transfer rate standard test ASTM E96M-05. This layer may have an air permeability from 10 seconds/100 ml to about 100 seconds/100 ml as measured, for example, by the Gurley method, and conforming to the STM D737 and WSP 70.1 standards. It may have a weight per unit area in the range of about 12 grams per square meter (gsm) to about 20 gsm and a thickness of about 35 to about 2000μ, in some cases from 80 to about 350μ. In some embodiments, the layer 208 is a calcium carbonate 40%/polyethylene 60% film stretched in the machine direction to enhance breathability. Beneath the film layer 208 is the spunbond backing layer 210, which will be discussed in more detail below.

As indicated above, the illustrated structure includes three layers of adhesive, namely layers 202, 207, and 209. Suitable adhesive materials useful in these layers include, but are not limited to, hot melt adhesives, drying adhesives, contact adhesives, UV curing adhesives, light curing adhesives, and pressure sensitive adhesives, including for instance animal glue, collagen-based glue, albumin glue, casein glue, Canada balsam, gum Arabic, latex, methyl cellulose, library glue, mucilage, resorcinol resin, starch, urea-formaldehyde resin, acrylonitrile, cyanoacrylate, acrylic, epoxy resins, epoxy putty, ethylene-vinyl acetate, phenol formaldehyde resin, polyamide, polyester resins, polyethylene, polypropylene, polysulfides, polyurethane, polyvinyl acetate, polyvinyl alcohol, polyvinyl chloride, polyvinyl chloride emulsion, polyvinylpyrrolidone, rubber cement, silicones, styrene acrylic copolymer, ethylene-acrylate copolymers, polyolefins, atactic polypropylene, polybutene-1, oxidized polyethylene, styrene block copolymers, polycarbonates, fluoropolymers, silicone rubbers, or the like and various other co-polymers. The adhesive may further comprise one or more additives including, but not limited to, tackifying resins, waxes, plasticizers, antioxidants, stabilizers, UV stabilizers, pigments, dyes, biocides, flame retardants, antistatic agents, and fillers or the like. The adhesive layer(s) can be continuous or applied in various patterns, and may contact only a portion of, or substantially the entire surface area of any two layers adhered together. The same or different adhesives may be used in each layer.

The foregoing drypad of FIGS. 2 and 3 is exemplary and it is contemplated that the spunbond backing material described herein may be used in connection with any other drypad, underpad, or other bed pad structure for absorbing moisture emissions from a patient. In another general configuration, the bed pad may be composed of first and second sublayers, wherein the first sublayer comprises at least one of polypropylene, polyethylene, polyethylene terephthalate, polyamide, viscose rayon, nylon, and starch-based biodegradable material. The second sublayer may comprise at least one of polyethylene, polypropylene, perforated polyethylene, perforated polypropylene, polylactic acid), polyhydroxybutyrate, and combinations thereof. The bed pad may take the form of an underpad or a drypad, and may have one, two, three, four, or more than four layers of absorbent material in the absorbent structure that is connected to the backing layer.

Generally, irrespective of the composition of the absorbent structures in the bed pad, the bed pad is provided with a backing layer that comprises a composite material comprising a first polymeric fiber and a second polymeric fiber, wherein the second polymeric fiber being relatively tacky relative to the first polymeric fiber and wherein the second fiber is present in said backing layer in an amount relatively smaller than the amount of the first fiber. Preferably, the second fiber comprises a bicomponent fiber. While various structures are contemplated for the backing layer, the backing layer may be a heterogenous spunbond material in which the second polymeric fiber is disposed predominantly on an exterior face of the bed pad.

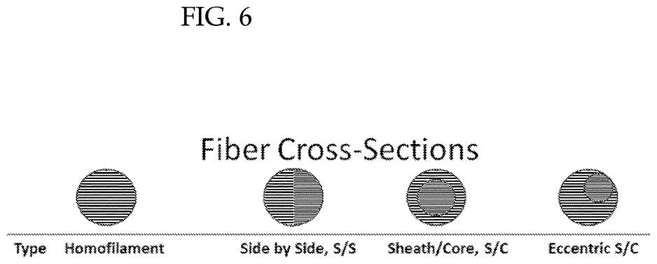

Bicomponent fibers, also known as conjugate fibers, generally comprise fibers containing multiple discrete polymers, especially extruded fibers wherein multiple polymers are extruded from the same spinneret. Various fiber constructs are schematically represented in FIG. 6, including homofilament fibers and including three varieties of bicomponent fibers. Although either the side-by-side fiber or eccentric fiber construct may be useful in some embodiments, in practice a sheath/core fiber is preferred. The sheath/core fiber has a core polymer that is essentially completely enveloped by the sheath polymer and, for this reason, the surface tackiness of the fiber is dictated by the properties of the sheath polymer. This allows the producer to use a more inexpensive material for the core polymer and to reserve the tackier, and typically more expensive, material for the sheath polymer.

Any suitable materials can be used for the sheath and core polymers. In one embodiment, the core material is polypropylene and the sheath material is a copolymer of polypropylene with ethylene repeat units. Exemplary polymers suitable as the sheath polymer as are sold under the trademarks VERSIFY by Dow Corp. and VISTAMAX ExxonMobil Corp. These polymers generally comprise isotactic propylene repeat units with random ethylene distribution. The polymer alternatively may be a block copolymer, such as one of the INFUSE polymers, also available from Dow Corp. Generally, any polymer that contains two domains, one more rigid than the other, may be employed as the sheath polymer. Other such polymers include L-MODU, available from Idemitsu Kosan Co., Ltd. and AFFINITY, available from Dow Corp.

When the sheath polymer contains ethylene as the relatively less rigid polymeric domain, the ethylene content of the polymer may be any content suitable to provide tackiness to the fiber, and the amount employed may vary depending on the diameter of the fibers. For bicomponent fibers having a diameter of 15-35 microns, which is a useful fiber size, the ethylene content may be in the range of 10-20% by mass of the total content of the sheath polymer, such as about 15%. The ethylene content may be increased if larger fiber diameters are employed or may be decreased if smaller diameter fibers are employed.

Desirably, the coefficient of static friction of the backing layer, measured with reference to an acrylic sheet, is at least 110%, preferably at least 120%, more preferably at least 130%, more preferably at least 140%, more preferably at least 150%, more preferably at least 160%, more preferably at least 170%, more preferably at least 180%, more preferably at least 190%, and more preferably at least 200%, of the coefficient of static friction of a similar backing layer composed only of polypropylene. While conventional bedding material is not made of acrylic, it is believed that a backing layer that meets this criterion will have a beneficial anti-slip property when used on conventional bedding materials. Similarly, it is desirable that the coefficient of kinetic friction of the backing layer, measured with reference to an acrylic sheet, is at least 110%, preferably at least 120%, more preferably at least 130%, more preferably at least 140%, more preferably at least 150%, more preferably at least 160%, more preferably at least 170%, more preferably at least 180%, more preferably at least 190%, and more preferably at least 200%, of the coefficient of dynamic friction of a similar backing layer composed only of polypropylene. The coefficients of static and dynamic friction may be measured according to any suitable method, such as ASTM D1894.

Similarly, any suitable amount of the sheath polymer may be used relative to the core polymer in the bicomponent fiber. For example, the content of sheath polymer may range from 10-20% by mass of the bicomponent fiber, such as 15% by mass. Generally, any suitable content sufficient to provide tackiness may be employed. The fiber alternatively may be a homofilament composed entirely of the ethylene/propylene copolymer, although at present this is less desirable for reasons of cost and processability.

Such bicomponent fiber is used to prepare a nonwoven, preferably spunbond material that serves as the backing layer for a bed pad containing the absorbent structure described above. In some embodiments, the backing material may be homogeneous and may comprise solely the bicomponent fiber. In other embodiments, the backing material is a heterogenous spunbond material in which the second polymeric fiber is disposed predominantly on an exterior face of the bed pad. By this is contemplated that the backing material is not uniform across its thickness such that the bicomponent fiber is predominantly on one face of the material, and that face is oriented outwardly in the bed pad so as to be positionable adjacent a bedsheet or other bedding material.

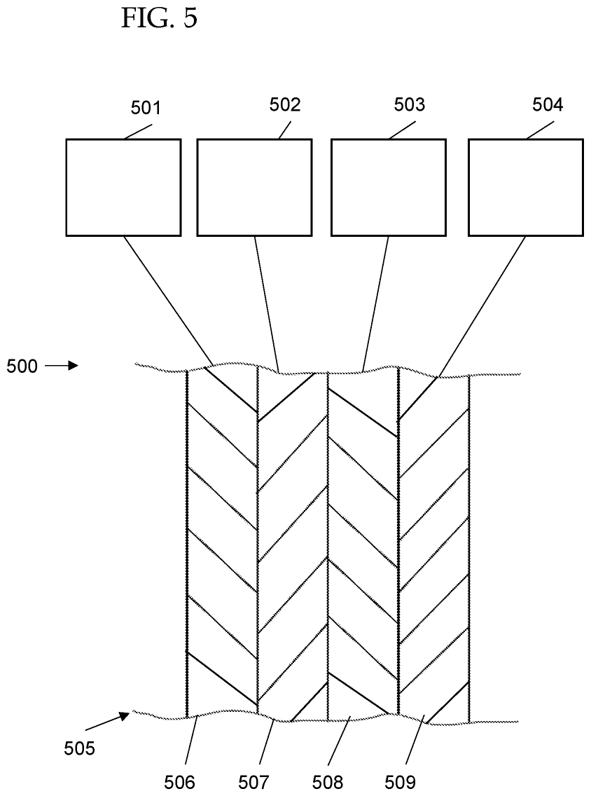

For example, with reference to FIG. 5, the exemplary spunbond processing line 500 includes banks 501, 502, 503, and 504. Processing line 500 may be any suitable spunbonding processing equipment, such as a REICOFIL R1, R2, R3, R4, or R5 machine. Banks 501-504 cooperate to produce a spunbond composite fabric 505, the fabric composed of four layers 506, 507, 508, and 509. In practice the spunbond process will not form entirely discrete layers 506-509, but rather, these layers will be somewhat intertwined and interconnected as is typical of spunbond fabric.

While it is possible to load bicomponent fiber for ejection from each of the banks 501-504, it will be more economical to load a homofilament, such as polypropylene, into three of the banks (501-503 or 502-504), reserving one of the terminal banks (504 or 501 respectively) for the bicomponent fiber. This will cause the formation of a heterogenous spunbond material in which the bicomponent fiber predominates on one face of the material. In the illustrated embodiment, four banks are shown, but a greater or smaller number of banks may be employed, and in any case it remains desirable for the bicomponent fiber to be loaded into one of the terminal banks.

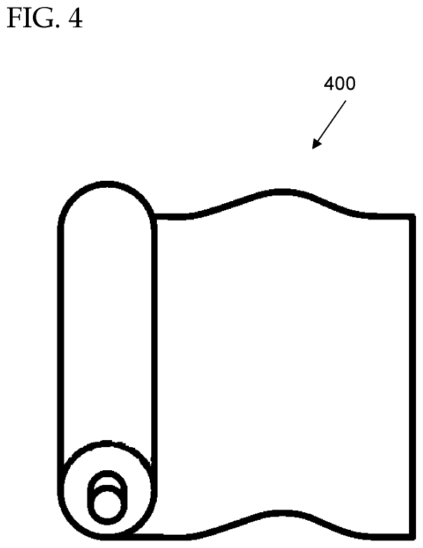

Once the backing layer has been prepared, it may be formed into a roll, such as roll 400 shown in FIG. 4. From this point, the absorbent bed pad may be made by any suitable method. Preferably, rolls of each layer to be incorporated into the absorbent pad 200 are positioned for lamination in conjunction with the superabsorbent polymer and adhesives, and after lamination via conventional equipment the laminar structure is then cut into sheets of the appropriate size. The bicomponent polymeric fiber will disposed predominantly on one face of the composite backing material, which will become the exterior face of the bed pad. The method thus includes joining the absorbent material and the optional other layers of the bed pad to the other face of the composite material, which is then designated the interior face. If necessary for finishing, any suitable edge bonding or finishing technique may be employed to finish the bed pads.

Alternatively, sheets of a suitable size are cut from rolls of each of the materials and laminated in sheet form. The process may be characterized as providing a roll of absorbent material, providing a second roll comprising the composite backing material, described herein, and joining the absorbent material to the composite material to form an absorbent structure. In practice, multiple rolls of material may be combined in a single operation or in stages to form a multi-layer laminar structure.

In use, a bed pad having the backing substrate prepared as described herein may be placed beneath a patient in and positioned to absorb fluids emanating from the patient, such as urine. The method may further comprise allowing one or more bodily fluids emitted by the patient to absorb into the absorbent structure. Optionally, the method may further comprise removing a soiled bed pad. Thereafter the method may further comprise replacing the soiled bed pad with a fresh pad. The bed pad will be less prone to slippage or bunching from patient movement than a conventional bed pad with a polypropylene backing.

The bed pad alternatively may be used in connection with a method for patient transfer and/or turning. For example, a patient transfer device known as the COMFORT GLIDE is sold by Medline Industries, Inc. This device has a relatively low-friction side and a relatively high-friction side, and the device may be used in connection with patient transfer. A bed pad prepared in accordance with the present teachings may be placed over a patient transfer device to absorb bodily fluids excreted by a patient before or after patient transfer. The bed pad also may be used with patient positioning devices that employ wedge devices having a relatively low-friction inclined surface, where a patient is placed on the device and the positioning device is used to slide the patient up the inclined surface of the wedge. This arrangement may be used to attempt to mitigate pressure ulcers in immobile patients, where the bed pad is again placed between the patient and the positioning device to absorb bodily fluids.

Uses of singular terms such as “a,” “an,” are intended to cover both the singular and the plural, unless otherwise indicated herein or clearly contradicted by context. The terms “comprising,” “having,” “including,” and “containing” are to be construed as open-ended terms. Any description of certain embodiments as “preferred” embodiments, and other recitation of embodiments, features, or ranges as being preferred, or suggestion that such are preferred, is not deemed to be limiting. The invention is deemed to encompass embodiments that are presently deemed to be less preferred and that may be described herein as such. All methods described herein can be performed in any suitable order unless otherwise indicated herein or otherwise clearly contradicted by context. The use of any and all examples, or exemplary language (e.g., “such as”) provided herein, is intended to illuminate the invention and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims. Neither the marking of the patent number on any product nor the identification of the patent number in connection with any service should be deemed a representation that all embodiments described herein are incorporated into such product or service.

1. A bed pad comprising:
a liquid-absorbent structure; and
a composite nonwoven backing layer comprising a first polymeric fiber and a second polymeric fiber, the second polymeric fiber being relatively tacky relative to the first polymeric fiber and being present in said backing layer in an amount relatively smaller than the amount of said first fiber and comprising a bicomponent fiber,
the coefficient of static friction of the backing layer, measured with reference to an acrylic sheet, being at least 110% of the coefficient of static friction of a similar backing layer composed only of polypropylene.

2. The bed pad of claim 1, the backing layer comprising a heterogenous spunbond material in which the second polymeric fiber is disposed predominantly on an exterior face of the bed pad.

3. The bed pad of claim 2, wherein the first fiber comprises polypropylene and the second fiber comprises polypropylene with random ethylene repeat units.

4. The bed pad of claim 3, wherein the second fiber is of a sheath/core structure and comprises a polypropylene core material and a sheath material, the sheath material comprising polypropylene with random ethylene repeat units.

5. The bed pad of claim 4, wherein the sheath material comprises from 10-20% of the mass of the second fiber.

6. The bed pad of claim 4, wherein random ethylene repeat units comprise from 10-20% of the mass of the sheath material.

7. The bed pad of claim 1, the liquid-absorbing structure comprising at least first and second liquid-absorbent sublayers.

8. The bed pad of claim 7, wherein the first sublayer comprises at least one of polypropylene, polyethylene, polyethylene terephthalate, polyamide, viscose rayon, nylon, and starch-based biodegradable material.

9. The bed pad of claim 7, wherein the second sublayer comprises a cellulosic absorbent layer.

10. The bed pad of claim 7, the liquid-absorbent layer comprising a top sheet, at least one cellulosic absorbent sheet, and a superabsorbent polymer.

11. A method of absorbing fluids comprising:
placing the bed pad of claim 1 beneath a patient; and
allowing one or more bodily fluids emitted by the patient to absorb into the absorbent structure.

12-13. (canceled)

14. The bed pad of claim 1, wherein the coefficient of kinetic friction of the backing layer, measured with reference to an acrylic sheet, is at least 110% of the coefficient of dynamic friction of a similar backing layer composed only of polypropylene.

15. A bed pad comprising:
a liquid-absorbent structure; and
a composite nonwoven backing layer comprising a first polymeric fiber and a second polymeric fiber, the second polymeric fiber being relatively tacky relative to the first polymeric fiber and being present in the backing layer in an amount relatively smaller than the amount of the first polymeric fiber, and the second polymeric fiber comprising a propylene ethylene copolymer,
the coefficient of static friction of the backing layer, measured with reference to an acrylic sheet, being at least 110% of the coefficient of static friction of a similar backing layer composed only of polypropylene.

16. The bed pad of claim 15, wherein the coefficient of kinetic friction of the backing layer, measured with reference to an acrylic sheet, is at least 110% of the coefficient of dynamic friction of a similar backing layer composed only of polypropylene.

17. The bed pad of claim 15, wherein the second polymeric fiber is a homofilament.

18. A method of absorbing fluids comprising:
placing the bed pad of claim 15 beneath a patient; and
allowing one or more bodily fluids emitted by the patient to absorb into the absorbent structure.

19. A bed pad comprising:
a liquid-absorbent structure; and
a spunbond backing layer comprising polypropylene fiber and fiber comprising a propylene ethylene copolymer, the fiber comprising the propylene ethylene copolymer being tacky relative to the polypropylene fiber and being in the backing layer in an amount relatively smaller than the amount of the polypropylene fiber,
the coefficient of static friction of the backing layer, measured with reference to an acrylic sheet, being at least 110% of the coefficient of static friction of a similar backing layer composed only of polypropylene.

20. The bed pad of claim 19, wherein the coefficient of kinetic friction of the backing layer, measured with reference to an acrylic sheet, is at least 110% of the coefficient of dynamic friction of a similar backing layer composed only of polypropylene.

21. The bed pad of claim 19, wherein the fiber comprising the propylene ethylene copolymer is disposed predominantly on an exterior face of the bed pad.

22. A method of absorbing fluids comprising:
placing the bed pad of claim 19 beneath a patient; and
allowing one or more bodily fluids emitted by the patient to absorb into the absorbent structure.
